(19) United States
(12) Patent Application Publication
(10) Pub. No.: US 2018/0077894 A1
(43) Pub. Date: Mar. 22, 2018

(54) METHODS AND SYSTEMS FOR DETECTION AND TRACKING OF MASTITIS IN DAIRY CATTLE

(71) Applicant: SRC, Inc., North Syracuse, NY (US)

(72) Inventors: Timothy Francis Moshier, Fulton, NY (US); Huda S. Suliman, Liverpool, NY (US); Kenton Arthur Doctor, East Syracuse, NY (US); Jeffrey H. Mills, Chittenango, NY (US); Daryl V. Nydam, Freeville, NY (US); Bradley J. Rauch, Dryden, NY (US); Anja S. Sipka, Ithaca, NY (US); Deborah L. Plochocki, Marcellus, NY (US); Linsay E. Edinger, Truxton, NY (US)

(73) Assignee: SRC, Inc., North Syracuse, NY (US)

(21) Appl. No.: 15/712,750

(22) Filed: Sep. 22, 2017

Related U.S. Application Data
(60) Provisional application No. 62/398,146, filed on Sep. 22, 2016.

(51) Int. Cl. A01J 5/013; G01N 33/04; A01K 11/00; A01J 5/007; G06F 19/00

(52) U.S. Cl. CPC A01J 5/013; G01N 33/04; A01K 11/004; A01K 2227/101; A01K 11/003; G06F 19/3493; G06F 19/366; A01J 5/007

(57) ABSTRACT

A method for identifying mastitis in a dairy animal, the method including the steps of: (i) identifying a dairy animal suspected of being affected by mastitis; (ii) collecting a sample of milk from the identified dairy animal; (iii) analyzing the sample for the presence of one or more mastitic bacteria; (iv) analyzing the sample for the presence of one or more mastitic pathogens; (v) identifying, if the presence of one or more mastitic bacteria is indicated, the mastitic bacteria in the sample; and (vi) determining, based on the identified mastitic bacteria in the sample, a management decision for the dairy animal.

### CROSS-REFERENCE TO RELATED APPLICATION

This application claims priority to co-pending U.S. Patent Application Ser. No. 62/398,146, filed on Sep. 22, 2016, and entitled “Methods and Systems for Detection and Tracking of Mastitis in Dairy Cattle,” the entire disclosure of which is incorporated herein by reference.

### FIELD OF THE INVENTION

The present disclosure is directed generally to a method for detecting and tracking mastitis in dairy animals.

### BACKGROUND

As farming operations grow, particularly dairy operations, there is an accompanying requirement for accurate data critical to precision management. Efficient animal tracking and maintenance, for example, is essential for a dairy farm of any size. Among many other conditions and situations that require efficient animal tracking and maintenance, on-farm pathogen identification systems ensure accurate identification of infected and treated animals. Providing readily-available and actionable information to operation managers enables on-the-spot treatment and/or other remediation decisions.

Of foremost importance, for example, is the identification and tracking of mastitis, a name given to inflammation of udder tissue due to infection, which can be caused by a number of different pathogens. Mastitis affects about 25% of all dairy cows each year, and the economic impact of mastitis in the U.S. alone is estimated to be between $1 billion to $2 billion per year. Much of the cost is due to reduced milk production by the affected animals, discarded milk due to poor quality, and the presence of antibiotics in the milk, which cannot be sold. In the cases of contagious mastitis, farmers also have the costs associated with culling the infected animals and purchasing replacements, and the potential for infections spreading to new animals.

Mastitis identification and tracking, however, can be difficult. In existing identification and tracking operations, there is a significant time lag between the detection of mastitis and a therapy decision. Typical screening methods require at least 24 hours to receive results, and in the hands of non-specialists these screening methods can produce unclear results. When results are unclear, for example, a farmer may either send samples to another laboratory and/or start antibiotic treatment that may not be necessary. Indeed, in about 50% of mastitis cases the dairy animal will have already cleared the infection, or she may clear the infection without treatment in a few days, but the lack of actionable information results in guessing rather than informed decision-making.

Accordingly, there is a continued need in the art for methods and systems for improved detection and tracking of mastitis in dairy animals.

### SUMMARY OF THE INVENTION

The present disclosure is directed to an inventive method for mastitis detection and tracking in dairy animals. Various embodiments and implementations herein are directed to a method for analyzing a sample collected from a potentially mastitic cow, first with a broad assay to identify the presence of mastitic bacteria, then with one or more targeted assays to identify one or more particular genera or species of bacteria. The results, which are often obtained in two hours or less, are associated throughout the analysis with the dairy animal that provided the sample. Optional Bluetooth transponders, RFID, or other tracking methods can enable rapid localization of the animal.

According to an aspect is a computerized method for identifying a management outcome for a dairy animal. The method includes: (i) identifying a dairy animal suspected of being affected by mastitis; (ii) tagging the identified dairy animal with a tag comprising a unique identifier; (iii) collecting a sample of milk from the identified dairy animal, wherein the sample of milk is coded to be associated with the dairy animal's unique identifier; (iv) associating, using a herd management computing device in communication with a herd management database, the tag with the collected sample; (v) analyzing the sample for the presence of one or more mastitic bacteria; (vi) identifying, if the presence of one or more mastitic bacteria is indicated, the mastitic bacteria present in the sample; (vii) providing the results of the analyzing and/or identifying step to the herd management computing device; and (viii) identifying, using the herd management computing device and based at least in part on the identified mastitic bacteria in the sample, a management outcome for the dairy animal.

According to an embodiment, the tag comprises a GPS receiver, an RFID tag, and/or a Bluetooth transponder.

According to an embodiment, the sample is analyzed using PCR analysis.

According to an embodiment, the first analyzing step comprises analysis of the collected sample using PCR analysis comprising a primer pair configured to amplify a conserved genomic region of a plurality of different mastitic bacteria.

According to an embodiment, the first analyzing step comprises analysis of the collected sample using PCR analysis comprising a plurality of primer pairs each configured to amplify a unique genomic region of a particular mastitic bacteria species.

According to an embodiment, the analyzing step is further configured to determine whether mastitic bacteria in the sample are Gram positive or Gram negative.

According to an embodiment, the method further includes analyzing the sample for the presence of one or more mastitic pathogens.

According to an embodiment, the method further includes capturing an image of the identified animal and/or the tag.

According to an embodiment, the method further includes storing the results of the analyzing and/or identifying step in the herd management database.

According to an embodiment, the management outcome comprises treating the animal for the identified present mastitic bacteria, isolating the animal from a herd, and/or culling the animal from the herd.

According to an embodiment, the herd management computing device is a handheld computing device.

According to an embodiment, the sample is analyzed using PCR machine, and wherein the PCR machine is in communication with the herd management computing device.

According to an aspect is a system for identifying a management outcome for a dairy animal. The system includes: a herd management computing device configured to: (i) associate a tag, comprising a unique identifier associated with a dairy animal suspected of being affected by mastitis, with a code associated with a sample of milk from the identified dairy animal; and an analytical machine configured to: (i) analyze the collected sample of milk for the presence of one or more mastitic bacteria; (ii) analyze collected sample of milk for the presence of one or more mastitic pathogens; and (iii) identify, if the presence of one or more mastitic bacteria is indicated, the mastitic bacteria in the sample; where the herd management computing device is further configured to identify, based at least in part on the identified mastitic bacteria in the sample, a management outcome for the dairy animal.

According to an embodiment, the herd management computing device is a handheld device. According to an embodiment, the analytical device is a PCR machine.

According to an embodiment, the system further includes a herd management computing database configured to store an association between the tag and the code associated with the sample of milk from the identified dairy animal.

According to an embodiment, the herd management computing device is further configured to localize the identified dairy animal.

According to an embodiment, the herd management computing device comprises a camera, and the herd management computing device is further configured to associate an image of the identified dairy animal with one or both of the tag and the code associated with the sample of milk from the identified dairy animal.

According to an aspect is a herd management device. The herd management device includes: a processor configured to: (i) associate a tag, comprising a unique identifier associated with a dairy animal suspected of being affected by mastitis, with a code associated with a sample of milk from the identified dairy animal; (ii) receive, from an analytical machine, results of a first analysis of the collected sample of milk comprising a determination of a presence of one or more mastitic bacteria; (iii) receive, from the analytical machine, an identification, if the presence of one or more mastitic bacteria is indicated, the mastitic bacteria in the sample; (iv) identify, based at least in part on the identified mastitic bacteria in the sample, a management outcome for the dairy animal.

According to an embodiment, the device further includes a camera, and the processor is further configured to associate an image of the identified dairy animal with one or both of the tag and the code associated with the sample of milk from the identified dairy animal.

These and other aspects of the invention will be apparent from the embodiments described herein.

### BRIEF DESCRIPTION OF THE DRAWINGS

The present invention will be more fully understood and appreciated by reading the following Detailed Description in conjunction with the accompanying drawings, in which:

FIG. 1 is a flowchart of mastitis identification and tracking method, in accordance with an embodiment.

FIG. 2 is a schematic representation of a system for dairy animal mastitis identification and tracking, in accordance with an embodiment.

FIG. 3 is a representation of a method of mastitis identification and tracking, in accordance with an embodiment.

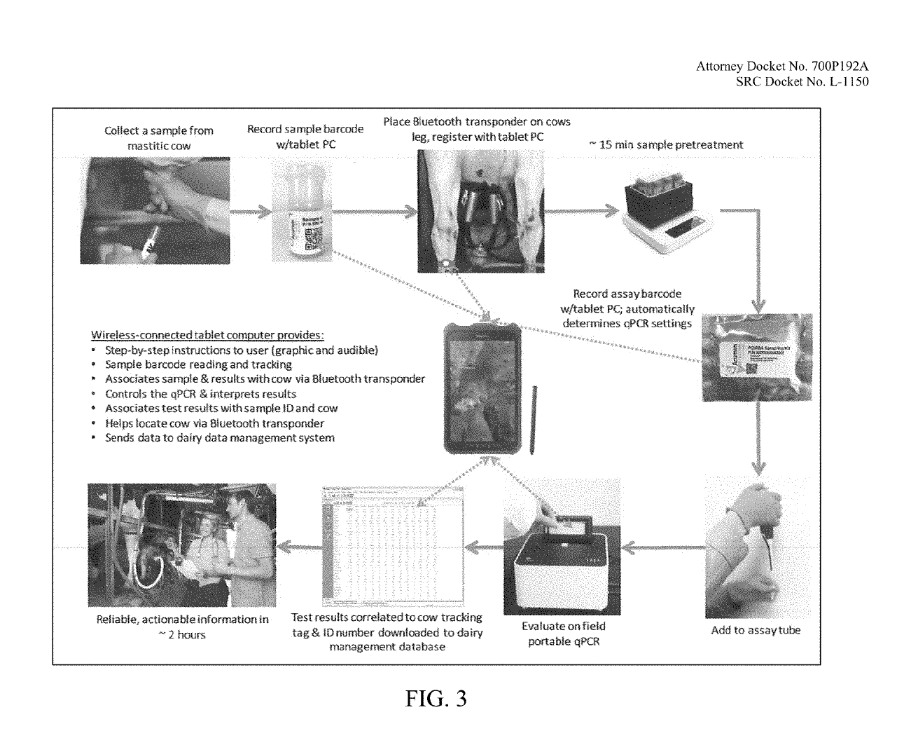

FIG. 4 is a representation of a method of mastitis identification and tracking, in accordance with an embodiment.

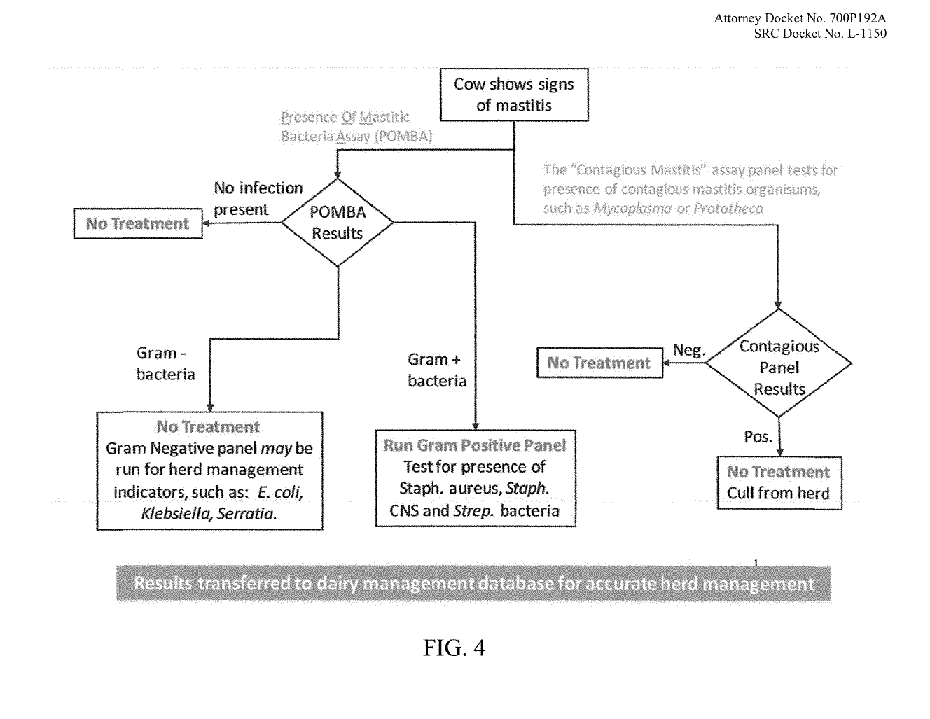

### DETAILED DESCRIPTION

The present disclosure describes various embodiments of a system and method for mastitis detection and tracking in dairy animals. Various embodiments and implementations herein are directed to a method for obtaining a sample from a potentially mastitic cow, and analyzing the sample with a broad assay to identify the presence of mastitic bacteria followed by one or more targeted assays to identify one or more particular genera or species of mastitic bacteria. The system and method associates the sample and results with the dairy animal that provided the sample, and may optionally provide a mechanism for localizing the animal if treatment or management is necessary.

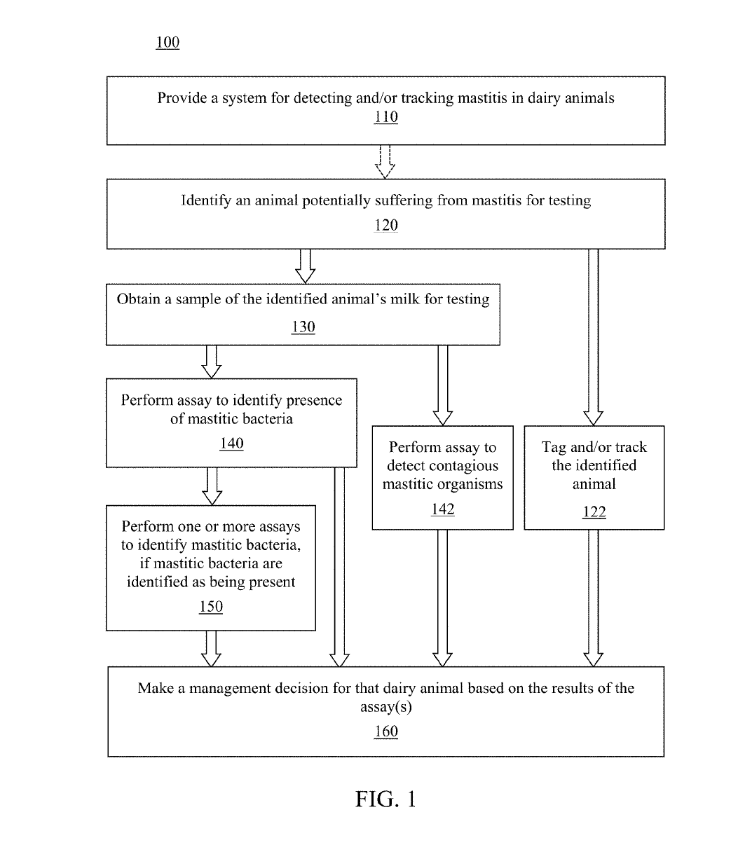

Referring to FIG. 1 is a schematic representation of a method 100 for mastitis detection and tracking, in accordance with an embodiment. At step 110 of the method, a system is provided to enable the detection and tracking of mastitis in dairy animals. The system may comprise one or many different components, and may be provided at one or many different locations. The system may be any of the systems described or otherwise envisioned herein.

At step 120 of the method, a dairy animal is identified for testing. Identification is typically based on visual inspection of the udder and/or expressed milk. For example, during milking time the dairy animal enters the milking parlor and backs into or walks into a milking stanchion. The milker will then visually inspect the udder to see if it is inflamed and/or express a small amount of milk to see if it shows signs of mastitis disease, such as watery or clotted milk. If it looks like the animal has mastitis, the animal will be identified for testing. According to another embodiment, animals are randomly identified for testing, and/or other methods are utilized to identify a dairy animal for testing.

At step 122 of the method, the identified animal is tagged or otherwise tracked. For example, among many other options, a leg band may be wrapped around the animal's leg and her milk will be separated from the rest of the collected milk. That cow will then go back to the herd, field, or barn, and it may be challenging to find which animal out of a herd of hundreds or more has the tag or leg band. As another example, the animal is tagged with a tag, collar, leg band, or other tagging component comprising a Bluetooth transponder, an RFID tag, a GPS tag, or any other trackable tag. According to one embodiment, the identified dairy animal is tagged with an identifier that is unique to the dairy animal, thus enabling subsequent identification. According to an embodiment, the tag also comprises a localization mechanism. For example, the tag may be an RFID tag that enables localization as an RFID scanner moves through the location where the animal is located, or as the animals move through an RFID scanner. As another example, the tag may comprise a GPS receiver and may transmit its location to a central server, computer, or other receiver. The tag may transmit its location periodically, continuously, or in response to a request for transmission.

At step 130 of the method, a sample of the animal's milk is obtained for testing, and is coded to be associated with the animal's unique identifier. For example, the milker that identified the animal for testing or received an identification of the animal for testing can express or otherwise remove a sample of milk from the animal's udder. The sample can be collected in any collection device, although in a preferred embodiment the collection device is barcoded, tagged, or otherwise marked to allow tracking. Additionally, the sample may be tracked in the system to be associated in memory, by number, or in any other means with the tag on the identified dairy animal. Associating the collected sample with the tag allows for rapid subsequent identification of the dairy animal. In an embodiment where the tag can be localized, it also allows for rapid localization of the dairy animal if necessary.

Once the sample is collected, it is ready for analysis. According to an embodiment, the sample is analyzed on the farm or is shipped to a laboratory. On the farm, for example, the sample may be analyzed by a handheld device, and/or in a laboratory situated within or on the farm. Alternatively, the sample may be carried, shipped, or otherwise transported to a laboratory for analysis.

At step 140 of the method, a broad assay is performed to identify the presence of mastitic bacteria in the sample. According to one embodiment, instead of identifying which specific mastitic bacteria are present, if any, this assay may determine only whether bacteria are or are not present in the sample. Although the assay may be quantitative in nature, this is not a necessary feature of the assay.

According to an embodiment, minimal sample preparation is required on a raw milk sample collected from the dairy animal. For example, the method can utilize a simple, two-tube, less-than-30 minute treatment process that combines sample acidification/lysis with a short (15 minutes or less) heating in one tube, and pH neutralization and chelation in a second tube to remove the majority of PCR inhibitors in the sample and make more of the target DNA available. This minimizes sample dilution, and so increases overall sensitivity. Many other methods of sample preparation are possible.

According to an embodiment, PCR analysis is utilized to identify the presence of one or more target mastitic bacteria in this initial broad assay, which may be quantitative PCR. For example, the PCR amplification may be targeted to one or more regions of the genome conserved among the target mastitic bacteria, which would minimize the diversity of primers or other components necessary for the PCR analysis. For example, conserved ribosome sequences are a potential target for amplification. As another example, the PCR amplification may be targeted to one or more unique regions of the genome for each of the target mastitic bacteria, which would increase the diversity of primers and other components necessary for the PCR analysis. Combinations of these two approaches are also possible. The PCR analysis may be, for example, real-time PCR analysis.

According to one embodiment, therefore, the analysis comprises analysis of the collected sample using PCR analysis comprising a primer pair configured to amplify a conserved genomic region of a plurality of different mastitic bacteria. According to another embodiment, the analysis comprises analysis of the collected sample using PCR analysis comprising a plurality of primer pairs each configured to amplify a unique genomic region of a particular mastitic bacteria species.

According to an embodiment, the broad assay determines whether the mastitic bacteria are Gram positive bacteria, Gram negative bacteria, or both Gram negative and Gram positive bacteria. As described in greater detail below, this will determine what further assays, if any are performed on the sample.

At step 142 of the method, an assay is performed to identify the presence of mastitic contagions in the sample. According to an embodiment, these mastitic contagions are not amenable to detection in the broad assay in step 140. Examples of mastitic contagions include, but are not limited to, Prototheca and Mycoplasma, among others. Contagious mastitic bacteria or pathogens may be especially important to identify quickly in order to avoid spreading the infection within the herd.

At step 150 of the method, if mastitic bacteria are present, one or more assays are performed to identify the genus and/or species of the bacteria. The broad assay at step 140 of the method may determine, for example, that the detected mastitic bacteria in the sample are Gram positive bacteria, Gram negative bacteria, or both Gram negative and Gram positive bacteria. Dairy animals will typically clear Gram negative infections in a few days, and thus may not require treatment with antibiotics. This would save the farmer both the cost of the treatments and the lost milk revenue. Examples of Gram negative bacteria include, but are not limited to, E. coli, Klebsiella, and Serratia, among many others.

According to an embodiment, if the presence of Gram positive bacteria is indicated, the system may recommend one or more subsequent assays to further classify the suspect pathogen. Examples of Gram negative positive include, but are not limited to, S. aureus, S. CNS, and Streptococcus spp, among many others. In the case of Gram positive infections, the farmer may choose to use a targeted antibiotic treatment, and/or to cull the cow from the herd.

According to an embodiment, PCR analysis is utilized to characterize the Gram positive bacteria in the sample. For example, the PCR amplification may be targeted to one or more unique regions of the genome for each of the target mastitic bacteria, although other approaches are possible. The PCR analysis may be, for example, real-time PCR analysis.

At step 160 of the method, a management decision is made regarding the dairy animal, based on the outcome of the one or more assays in step 150. According to an embodiment, the management decision is based on information from one or more of the broad assay of step 140, the contagious mastitic organism assay of step 142, and the targeted assay of step 150, among other possible sources of information.

The management decision can be any of a variety of different decisions, including but not limited to one or more of: (i) doing nothing if the infection is likely to already be cleared and/or is likely to be cleared quickly without treatment; (ii) treating the animal with an antibiotic or other treatment; (iii) isolating the animal from the herd for a period of time; and/or (iv) culling the animal from the herd permanently. Other management decisions are possible.

Accordingly, the method and accompanying system is a fully-integrated method and system that enables virtually anyone to successfully identify the presence of mastitic pathogens, on the farm within a couple of hours, and minimizes the opportunity for error. The system and method ensures a high level of fidelity between the dairy animal, the sample, and sample results.

According to an embodiment, the method comprises a herd management computing device configured to perform and/or facilitate one or more steps of the method. For example, the herd management computing device may associate the dairy animal's unique tag with the sample collected from the dairy animal. The herd management computing device may also inform an analytical device such as a PCR machine which analysis to perform, including one or more settings of the device. The herd management computing device may also receive the results of the analysis, and may store the results in a herd management database. The herd management computing device may also provide an output comprising a recommended management outcome for the dairy animal based at least in part on the results of an analysis of the sample, among other possible input. The herd management computing device may also facilitate location of the dairy animal using the unique identifier and/or tag associated with the animal. For example, the herd management computing device may comprise a locater such as a Bluetooth receiver, an RFID scanner, a transceiver to receive GPS information from a tag, or other methods to locate the dairy animal. Alternatively, the herd management computing device may be in communication with a device configured to facilitate localization of the animal, such as a Bluetooth receiver, an RFID scanner, a transceiver to receive GPS information from a tag. The herd management computing device may be configured to communicate with a centralized herd management computing system, computer, or server, and may receive and/or send information to a dairy data management system as described or otherwise envisioned herein. The herd management computing device may be any computing device, including but not limited to a handheld computing device such as a smartphone, laptop, tablet, wearable, or any other computing device.

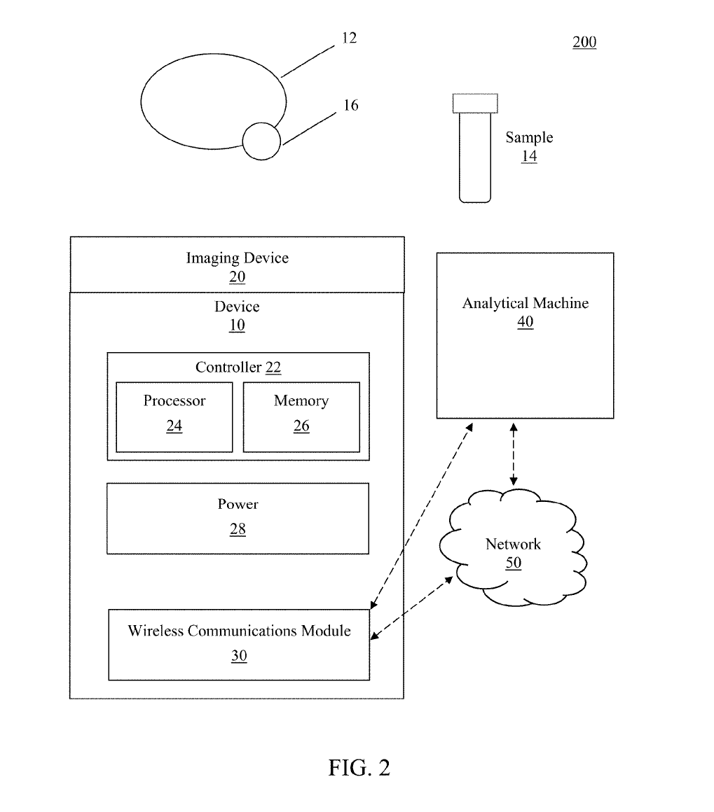

Referring to FIG. 2 is a schematic representation of a system 200 for mastitis identification and tracking, in accordance with an embodiment. According to an embodiment, system 200 is configured to analyze a sample obtained from an udder 12 of a dairy animal suspected to be affected by mastitis, where the dairy animal is any animal that provides milk used by humans, including but not limited to cow, buffalo, goat, sheep, camel, donkey, horse, reindeer, and yak.

According to an embodiment, system 200 includes a sample collection tube or device 14 that receives a sample of milk expressed from the identified dairy animal. The system also comprises a mounted, portable, or handheld device 10 that is utilized to receive or obtain information about the dairy animal and/or about the sample 14. For example, the device 10 may include an imaging device 20 such as a camera which is configured to capture one or more images of the sample 14, such as a barcode or other identifier. The imaging device may be connected to a controller 22, and transmits the captured image information to the controller and/or via a wireless communications module 30. The wireless communications module 30 can be, for example, Wi-Fi, Bluetooth, IR, radio, or near field communication that is positioned in communication with controller 22 or, alternatively, controller 22 can be integrated with the wireless communications module. Controller 22 can be configured or programmed to capture images of a sample 14 using imaging device 20. Controller 22 can be or have, for example, a processor 24 programmed using software to perform various functions discussed herein, and can be utilized in combination with a memory 26. Memory 26 can store data, including one or more captured images or software programs for execution by processor 24, as well as various types of data including but not limited to information about specific animals. For example, the memory 26 may be a non-transitory computer readable storage medium that includes a set of instructions that are executable by processor 24, and which cause the system to execute one or more of the steps of the methods described herein.

According to an embodiment, imaging device 20 may be configured to capture one or more images of or other information about a tag 16 on or about the identified dairy animal. This will allow for tracking of the animal, and allows for association of the animal and the collection device 14.

In addition to imaging the sample and tag 16, the device 10 may be configured to obtain information directly from a user. For example, the device 10 or an associated device may comprise a user input that allows the herdsman to enter information or make a selection about the animal. It may be a text entry field, a button, a swipe, a touch, or any other method of data entry or selection. For example, the user interface may request an input whenever an animal is identified that is healthy or possibly not-healthy, or otherwise requires tracking. Suspicion of mastitis or another condition may also be associated with the animal. This may trigger handling of the milk in a manner different from other animals, such as diverting it to a different collection or location.

Device 10 also includes a source of power 28, such as DC power sources, AC power sources, solar-based power sources, or mechanical-based power sources, among others. The power source may be in operable communication with a power source converter that converts power received from an external power source to a form that is usable by the lighting unit. In order to provide power to the various components of device 10, it can also include an AC/DC converter (e.g., rectifying circuit) that receives AC power from an external AC power source 28 and converts it into direct current for purposes of powering the light unit's components. Additionally, device 10 can include an energy storage device, such as a rechargeable battery or capacitor, that is recharged via a connection to the AC/DC converter and can provide power to controller 22 and imaging device 20 when the circuit to AC power source 28 is opened.

According to an embodiment, system 100 also comprises an analytical machine 40, such as a PCR machine, sequencer, and/or other device. Analytical machine 40 may be one or multiple machines. The device may be located on the farm or located remotely. According to an embodiment, device 10 may communicate with analytical machine 40 directly via a wired and/or wireless communications link, and/or via a wireless network 50.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The above-described embodiments of the described subject matter can be implemented in any of numerous ways. For example, some embodiments may be implemented using hardware, software or a combination thereof. When any aspect of an embodiment is implemented at least in part in software, the software code can be executed on any suitable processor or collection of processors, whether provided in a single device or computer or distributed among multiple devices/computers.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

What is claimed is:

1. A computerized method for identifying a management outcome for a dairy animal, the method comprising the steps of:
identifying a dairy animal suspected of being affected by mastitis;
tagging the identified dairy animal with a tag comprising a unique identifier;
collecting a sample of milk from the identified dairy animal, wherein the sample of milk is coded to be associated with the dairy animal's unique identifier;
associating, using a herd management computing device in communication with a herd management database, the tag with the collected sample;
analyzing the sample for the presence of one or more mastitic bacteria;
identifying, if the presence of one or more mastitic bacteria is indicated, the mastitic bacteria present in the sample;
providing the results of the analyzing and/or identifying step to the herd management computing device; and
identifying, using the herd management computing device and based at least in part on the identified mastitic bacteria in the sample, a management outcome for the dairy animal.

2. The method of claim 1, wherein the tag comprises a GPS receiver, an RFID tag, and/or a Bluetooth transponder.

3. The method of claim 1, wherein the sample is analyzed using PCR analysis.

4. The method of claim 1, wherein said first analyzing step comprises analysis of the collected sample using PCR analysis comprising a primer pair configured to amplify a conserved genomic region of a plurality of different mastitic bacteria.

5. The method of claim 1, wherein said first analyzing step comprises analysis of the collected sample using PCR analysis comprising a plurality of primer pairs each configured to amplify a unique genomic region of a particular mastitic bacteria species.

6. The method of claim 1, wherein said analyzing step is further configured to determine whether mastitic bacteria in the sample are Gram positive or Gram negative.

7. The method of claim 1, further comprising the step of analyzing the sample for the presence of one or more mastitic pathogens.

8. The method of claim 1, further comprising the step of capturing an image of the identified animal and/or the tag.

9. The method of claim 1, further comprising the step of storing the results of the analyzing and/or identifying step in the herd management database.

10. The method of claim 1, wherein the management outcome comprises treating the animal for the identified present mastitic bacteria, isolating the animal from a herd, and/or culling the animal from the herd.

11. The method of claim 1, wherein the herd management computing device is a handheld computing device.

12. The method of claim 1, wherein the sample is analyzed using PCR machine, and wherein the PCR machine is in communication with the herd management computing device.

13. A system for identifying a management outcome for a dairy animal, the system comprising:
a herd management computing device configured to: (i) associate a tag, comprising a unique identifier associated with a dairy animal suspected of being affected by mastitis, with a code associated with a sample of milk from the identified dairy animal; and
an analytical machine configured to: (i) analyze the collected sample of milk for the presence of one or more mastitic bacteria; (ii) analyze collected sample of milk for the presence of one or more mastitic pathogens; and (iii) identify, if the presence of one or more mastitic bacteria is indicated, the mastitic bacteria in the sample;
wherein the herd management computing device is further configured to identify, based at least in part on the identified mastitic bacteria in the sample, a management outcome for the dairy animal.

14. The system of claim 13, wherein the herd management computing device is a handheld device.

15. The system of claim 13, wherein the analytical device is a PCR machine.

16. The system of claim 13, further comprising a herd management computing database configured to store an association between the tag and the code associated with the sample of milk from the identified dairy animal.

17. The system of claim 13, wherein the herd management computing device is further configured to localize the identified dairy animal.

18. The system of claim 13, wherein the herd management computing device comprises a camera, and wherein the herd management computing device is further configured to associate an image of the identified dairy animal with one or both of the tag and the code associated with the sample of milk from the identified dairy animal.

19. A herd management device, comprising:
a processor configured to: (i) associate a tag, comprising a unique identifier associated with a dairy animal suspected of being affected by mastitis, with a code associated with a sample of milk from the identified dairy animal; (ii) receive, from an analytical machine, results of a first analysis of the collected sample of milk comprising a determination of a presence of one or more mastitic bacteria; (iii) receive, from the analytical machine, an identification, if the presence of one or more mastitic bacteria is indicated, the mastitic bacteria in the sample; (iv) identify, based at least in part on the identified mastitic bacteria in the sample, a management outcome for the dairy animal.

20. The herd management device of claim 19, further comprising a camera, and wherein the processor is further configured to associate an image of the identified dairy animal with one or both of the tag and the code associated with the sample of milk from the identified dairy animal.
